(19) United States
(12) Patent Application Publication
(10) Pub. No.: US 2004/0266659 A1
(43) Pub. Date: Dec. 30, 2004

(54) SUBSTANCES THAT ENHANCE RECALL AND LUCIDITY DURING DREAMING

(75) Inventor: Stephen P. LaBerge, Palo Alto, CA (US)

Correspondence Address: Mr. Mark D. Lenhart, 205 Robin Road, Burlingame, 94010-6633

(73) Assignee: Stephen P. LaBerge, Palo Alto (US)

(21) Appl. No.: 10/604,138

(22) Filed: Jun. 27, 2003

(51) Int. Cl. A61K 31/55; A61K 31/4745

(52) U.S. Cl. 514/1; 514/214.03; 514/291; 514/221

(57) ABSTRACT

A method of enhancing lucid dreaming comprising administration to individuals the Acetylcholine Esterase inhibitor class of drugs. Use of therapeutic agents recently developed for Alzheimer”s Disease such as Donepizil (AriceptÂ®), Rivastigmin (ExelonÂ®), Galantamine (ReminylÂ®, NivalinÂ®), and Huperzine results in minimal side-effects (e.g., insomnia and gastrointestinal symptoms) while significantly enhancing dreaming cognitive clarity, lucidity, self-reflection, recall, control, bizarreness, and visual vividness.

### BACKGROUND OF INVENTION

The term “lucid dreaming” was coined by Frederik van Eeden who used the word “lucid” in the sense of mental clarity. Lucidity usually begins in the midst of a dream when the dreamer realizes that the experience is not occurring in physical reality, but is a dream. Often this realization is triggered by the dreamer noticing some impossible or unlikely occurrence in the dream, such as flying or meeting the deceased. Sometimes people become lucid without noticing any particular clue in the dream; they just suddenly realize they are in a dream. A minority of lucid dreams (according to the research of LaBerge and colleagues, about 10 percent) are the result of returning to REM (dreaming) sleep directly from an awakening with unbroken reflective consciousness. The basic definition of lucid dreaming requires nothing more than becoming aware that you are dreaming. However, the quality of lucidity can vary greatly. When lucidity is at a high level, you are aware that everything experienced in the dream is occurring in your mind, that there is no real danger, and that you are asleep in bed and will awaken shortly. With low-level lucidity you may be aware to a certain extent that you are dreaming, perhaps enough to fly or alter what you are doing, but not enough to realize that the people are dream representations, or that you can suffer no physical damage, or that you are actually in bed.

In the circumstances described above, it is strongly desired in the art to develop a safe method of aiding dreamers attempts at more frequently achieving higher states of lucidity in their dreams. One class of preferred embodiments of this invention include neurotransmitter modulators such as Acetylcholine Esterase inhibitors.

A number of substances have been suggested to enhance the likelihood of lucid dreaming, from vitamins to prescription drugs. There are few good scientific studies to back such claims. Lucid dreaming is highly subject to the placebo effect; the belief that something will stimulate a lucid dream is very effective. Many prescription drugs as well as marijuana and alcohol alter the sleep cycle, usually by suppressing REM sleep. This leads to a phenomenon called “REM rebound,” in which a person experiences intense, long REM periods after the drug has worn off. This can manifest as nightmares or, possibly, as lucid dreaming, since the brain is highly active. Drugs in the LSD family, including psilocybin and tryptamines actually stimulate REM sleep (in doses small enough to allow sleep), leading to longer REM periods. The potentially dangerous and illegal nature of these drugs is not conducive to their use as lucidity enhancers.

It is known in the scientific literature that Acetylcholine and its agonist as well as Acetylcholinesterase and its inhibitors/antagonist may be involved with REM and sleeping (Amatruda et al 1975; Baghdoyan et al 1984; Gillin et al 1985; Velazquez-Moctezuma et al 1991; Wauquier et al 1985). J. A. Hobson, on page 202 of his 1988 book, “The Dreaming Brain” states: “Cholinergic brainstem mechanisms cause REM sleep and dreaming”. It has also been discovered that microinjection of the Acetylcholine agonist carbachol in the certain locations of the brain, such as the Pons, elicits extended periods of REM sleep, and that many of the neurons critical for REM sleep are responsive to Acetylcholine.

Neither Hobson, nor any other others, discuss the possibility of altering Cholinergic levels via Acetylcholine Esterase inhibitors as a means of enhancing dream recall and lucidity.

Historically, there are many cultures that have believed to have found naturally occurring substances that in some way alter dream consciousness. One such substance, Calea zacatechichi or “Dream Weed”, is, according to Lilian Mayagoitia, et al, in a 1986 Journal of Ethnopharmacology article, “a plant used by the Chontal Indians of Mexico to obtain divinatory messages during dreaming.” The neuro-active compounds of this herb are reported to cause thought to be sesquiterpene lactones, and as far as is currently known, unrelated to Acetylcholine Esterase inhibition or inhibitors.

Both ancient traditional medicines and modern popular speculation has assigned Oneirogenic, or “Dream-Inducing” properties to numerous herbs and other natural substances, including herbs like Valerian, Mugwort, Mullein, Kava Kava, Dittany of Crete, St. Johns Wort, Salvia Divinorum, Scutellaria Indica, Licorice Root, Vervain, Jasmine, Honeysuckle, Datura, Bee Pollen, Catnip, Hops, Sculicap, Mimosa, Lavender, Damiana, Withania Somnifera, Passionflower, Chamomile, Cardamom, Gotu Kola, Ginkgo Biloba, Ibogaine, Verbena, Rose, Cinnamon, Marigold, Nutmeg, Peppermint, Holly, Yarrow and Anise. Few if any of these have any scientific basis or support for these purported effects. Other supplements and/or drugs are also claimed to be linked to lucidity, including B-vitamins, Melatonin, DMAE, and the psychedelic DXM. Some of these drugs and herbs may be addictive, poisonous and/or illegal.

Lotsof, in U.S. Pat. No. 4,499,096, issued Feb. 12, 1985, teaches a “Rapid method for interrupting the narcotic addiction syndrome” via the drug Ibogaine. According to the patent, this drug induces “dream enhancement or hallucinatory effect”. This claimed “oneirogenic” effect is due to the fact that it induces dream behavior with the ego perspective relatively intact. This is, as the patent mentions, more of a hallucinatory effect, not a lucid dreaming effect. Because of these hallucinatory and other side effects, however, this drug is illegal in the U.S., and is thus not a desirable means for enhancing sleep dream quality. This drug is also not considered to be related to Acetylcholine Esterase inhibitors or ‘smart drugs’.

DMAE (2-dimethylaminoethanol) is a chemical that has been suggested by used to treat a number of conditions affecting the brain and central nervous system. It is thought to work by increasing production of the neurotransmitter Acetylcholine, although this has not been proven. Marketed as a memory and mood enhancer, DMAE is believed to enhance intellectual functioning. There are few good clinical studies supporting this belief. Such substances are known as “cholinergics” because of the belief that they increase Acetylcholine. They have been traditionally used to treat diseases such as Alzheimer's dementia, tardive dyskinesia, and Huntington's chorea. Because DMAE is believed to be a cholinergic, it has been suggested for these disorders, even though placebo-controlled studies have provided essentially negative results. There is continued controversy over whether DMAE actually increases Acetylcholine. Nonetheless, Sergio, W. Claims in the 1988 August edition of Medical Hypotheses, in the article: “Use of DMAE (2-dimethylaminoethanol) in the induction of lucid dreams”, subjective experiences of himself and his wife experiencing enhanced lucidity through use of this supplement. It is unclear from the article to what extent his purported results derive from placebo response or any specific or cholinergic effect.

Also well known in the art is U.S. Pat. No. 5,507,716, awarded to LaBerge, et al. on Apr. 16, 1996, for Equipment and methods used to induce lucid dreams in sleeping persons. In this patent, a device was employed to assist people to achieve lucid dreams via the detecting and monitoring the eye and head movements of sleeping persons, where eye movement activity in the absence of head movement is used to indicate the presence of REM sleep. By then applying sensory stimuli to sleepers in REM sleep can cue them that they are dreaming without producing awakening. Other persons have developed equipment, such as Keith M. T. Hearne who illustrated and described his respiratory measuring device in his 1983 U.S. Pat. No. 4,420,001. His device sensed temperature changes of a person's respiration in his or her breathing passageway, or in airflows to and from his or her breathing passageway. Thermistors were used, in an electrical circuit, to sense the temperature changes of the person's respiration. When the rate of these temperature changes reached a high predetermined level, the signals created in the electrical circuit initiated an audible sound, either to help arouse a sleeping person from an unpleasant dream by awaking them or to help them enter into a lucid dream state.

None of the preceding references disclosed describe a method of enhancing lucid dreaming comprising administering to individuals the Acetylcholine Esterase inhibitor class of drugs. Use of therapeutic agents for Alzheimer”s Disease such as Donepizil (AriceptÂ®), Rivastigmin (ExelonÂ®), Galantamine (ReminylÂ®, NivalinÂ®), Tacrine and Huperzine leads to low incidence of adverse events, such as insomnia and gastrointestinal symptoms, while significantly enhancing dreaming cognitive clarity, lucidity, self-reflection, recall, control, bizarreness, and visual vividness.

### SUMMARY OF INVENTION

This invention relates to the field of Lucid Dreaming and the enhancement of dream recall and dream lucidity through memory enhancing drugs, including the class of substances that comprise Acetylcholine Esterase inhibitors (AChEls). Lucid dreaming involves dreaming while knowing that you are dreaming. AChE inhibitors (AchEl's) inhibit the normal metabolic inactivation of Acetylcholine (ACh) by inhibiting the enzyme, Acetylcholine Esterase (AChE), leading to accumulation of Ach. AchEl's are most commonly used to enhance memory, particularly in patients suffering from Alzheimer's disease. Ach is also well known to be important in REM and thus is suggested herein to enhance dreaming and lucidity.

### DETAILED DESCRIPTION

While investigating the efficacy of donepizil (AriceptÂ®) as a means to enhance lucid dreaming and cognitive clarity during REM sleep in normal subjects, the following experiment was performed with the following results: A randomized, double blind, placebo-controlled crossover trial was performed with ten normal volunteers self-selected for high dream recall and interest in lucid dreaming (7 male, 3 female, age 22-55). Subjects collected dream content and other self-report measures on three nights, separated by a washout period of at least one week. At bedtime, they took capsules containing 0 (placebo), 5, or 10 mg of donepizil, with counter-balanced order of the three dosages. After each spontaneous awakening during the three nights, Ss evaluated dream content on a range of measures including bizarreness, complexity, and intensity, affect, cognitive clarity, lucidity, and control. They also estimated sleep quality, insomnia, and degree of any adverse effects.

RESULTS: Nine of the 10 Ss (90%) reported one or more lucid dreams on the experimental nights, with only one S reporting a lucid dream on a placebo night. Cognitive clarity, lucidity, recall, control, bizarreness, and visual vividness were all significantly elevated with donepizil compared to placebo. The effects were generally dose-related with 10 mg donepizil producing significantly higher levels of these variables than the 5 mg dose, which in turn produced significant elevations compared to placebo. Odds of lucid dreaming for the three conditions increased from 0.031 for placebo, to 0.429 for 5 mg donepizil, and 0.754 for 10 mg donepizil. The lucidity odds ratio for the 10 mg dose compared to placebo was 24.3 (p<0.001). Donepizil was also associated with a significantly elevated frequency of sleep paralysis and a 40% increase in estimated time awake during the night (176 vs. 126 min of a 9.0 hr night, p<0.05). The incidence of adverse events, notably mild insomnia and gastrointestinal symptoms, was low and primarily associated with the higher dose condition (with two subjects reporting nausea and one vomiting).

Thus, compared to placebo, both 5 and 10 mg dosages of donepizil significantly enhance self-reported cognitive clarity, self-reflection, and lucidity during dreaming. These effects may be further enhanced by combination of the acetylcholinesterase inhibitors with Acetylcholine precursors, agonists and/or lucidity inducing electronic devices.

In addition to donepizil data, we have data on ExelonÂ® (rivastigmin; 6-12 mg dose) and galantamine (ReminylÂ®, NivalinÂ®8-16 mg) as well. Both work as well as AriceptÂ®), but with perhaps fewer side effects. We have also tested huperzine with promising results, but haven't yet worked out the dose correspondence with AriceptÂ® (but 5 mg Donepizil is more potent than 150 Apg huperzine).

In addition, we have reports of success with nicotine patches. There is also in the scientific literature studies showing triggering/intensification of REM sleep with arecoline. How much evidence do we need for these general claims: It should be noted that the REM altering, lucidity enhancing effects may be caused by a number of neurophysiological effects induced by these “smart drugs” besides Acetylcholinesterase inhibition. These include: 1. Using a cholinergic agonist (e.g., nicotine).

2. Using muscarinic receptor agonist: Drugs that mimic the effect of ACh on muscarinic receptors: Inc: muscarinic (M1), M2 and nicotinic Agonists. (Such as arecoline or Recoline, a muscarinic receptor agonist)₃. Using an antagonist of presynaptic receptors to activate the remaining Acetylcholine neurons. (i.e. In rats, antisense oligonucleotide sequences that block Muscarinic M2 (but not M4) receptors increases extracellular Acetylcholine. This effect (Antisense nucleotide sequences are complementary to a sequence of messenger RNA. When antisense DNA or RNA is added to a cell, it binds to a specific messenger RNA molecule and inactivates it.) 4. Using allosteric modulators (such as allosterically potentiating ligands [APLs]) of Acetylcholine and nicotinic receptors (drugs that interact with the receptor through binding sites that are distinct from those for Acetylcholine and nicotinic agonists and antagonists).

Other strategies for potentiating Acetylcholine function for dream lucidity enhancement, including the use of other classes of compound working in similar ways should be apparent to those skilled in the art from a consideration of the enclosed descriptions.

1. A method of enhancing the frequency and intensity of lucidity in dreaming comprising:
administering to an individual any of a class of substances that intensify REM sleep via the Acetylcholine esterase inhibitor class of drugs.

2. A method according to claim 1 wherein said inhibitor is selected from the group consisting of physostigmine, tetrahydroaminoacridine, tacrine, citicoline, velnacrine maleate, metrifonate, and heptastigmine, heptylphysostigmine, rivastigmine, donepezil, proserin, neostygmin, galantamine, nivalin, rivastimin, galantamine, codein, oxazyl, and Ambenonium chlorobenzylchloride.

3. A method according to claim 1 wherein said drug is administered at bedtime.

4. A method according to claim 1 wherein said method includes the simultaneous use of an electronic lucidity inducing device.

5. A method of enhancing the frequency and intensity of lucidity in dreaming comprising the step of administering to an individual any of a class of substances that intensify REM sleep;
said class selected from the group consisting of Acetylcholine esterase inhibitors, cholinergic agonists, Muscarinic receptor agonists, antagonist of presynaptic acetylcholine receptors, and allosteric modulators (such as allosterically potentiating ligands) of acetylcholine and nicotinic receptors.

6. A method according to claim 5 wherein said substances are selected from the group consisting of Nicotine, Pilocarpine, Pilocarpine Hydrochloride, Arecoline, Recoline and Carbachol.

7. A method according to claim 1 wherein said allowing time for REM sleep to be intensified, whereby lucid dreaming is enhanced.

8. A method of enhancing the frequency and quality of lucidity in dreaming, the method comprising administering an amount of a acetylcholine esterase inhibitor containing a composition sufficient to provide the dream enhancing benefit.

9. A method of enhancing lucid dreaming comprising:
administering to an individual at bedtime any of a class of substances that intensify REM sleep, including AChEIs (e.g., donepizil, rivastimin, galantamine), cholinergic agonists (e.g., nicotine), and other classes of compound working in similar ways, allowing time for REM sleep to be intensified, whereby lucid dreaming is enhanced.

10. A method of enhancing lucid dreaming comprising:
applying methods and procedures that intensify REM sleep, including total sleep deprivation, and selective REM deprivation whether produced through behavioral manipulation (e.g., REM dependent awakening) or drugs suppressing REM sleep causing subsequent rebound (e.g, barbiturates and amphetamine derivatives), allowing time for REM sleep to be intensified, whereby lucid dreaming is enhanced.
